(19) United States
(12) Patent Application Publication
(10) Pub. No.: US 2010/0009380 A1
(43) Pub. Date: Jan. 14, 2010

(54) COMPOSITIONS AND METHODS FOR CHARACTERIZATION OF CYSTEINE OXIDATIVE STATES

(75) Inventor: Kate S. Carroll, Ann Arbor, MI (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/496,751

(22) Filed: Jul. 2, 2009

Related U.S. Application Data
(60) Provisional application No. 61/077,654, filed on Jul. 2, 2008.

(51) Int. Cl. G01N 33/53; C07C 245/00; G01N 33/566

(52) U.S. Cl. 435/7.1; 534/550; 436/501

(57) ABSTRACT

The present invention relates to compositions and methods for characterization of cysteine oxidative states. In particular, the present invention provides cysteine-oxidative-state-specific labeling agents and uses thereof.

The present invention claims priority to U.S. Provisional Patent Application Ser. No. 61/077,654, filed Jul. 2, 2008, the disclosure of which is herein incorporated by reference in its entirety.

### FIELD OF THE INVENTION

The present invention relates to compositions and methods for characterization of cysteine oxidative states. In particular, the present invention provides cysteine-oxidative-state-specific labeling agents and uses thereof.

### BACKGROUND

Cysteine has been found to occur in up to 10 different sulfur oxidation states in vivo. Among these, the thiol (R—SH) and disulfide (R—S—S—R) oxidation states are the best known, but post-translational modifications such as sulfenic, (R—OH), sulfinic (R—SO₂H), and sulfonic (R—SO₃H) acids also play important roles in biochemistry. Despite studies implicating post-translational oxidative thiol modification as a modulator of cellular processes, the molecular details of the majority of these modifications, including the repertoire of proteins containing cysteine post-translational oxidative thiol modifications and the specific sites of modification, remain largely unknown.

Reactive oxygen species (ROS) are intracellular second messengers that regulate many normal cellular functions and are also implicated in the initiation and progression of human pathologies including arteriosclerosis, stroke, cancer, and aging. Redox-signaling pathways rely on protein “sensors” to convert the ROS signal into a specific cellular response. This process is mediated by the oxidation of thiols on specific cysteine residues to sulfenic (Cys-SOH), sulfinic (Cys-SO₂H) and sulfonic acids (Cys-SO₃H), which activates or deactivates their cellular function. Despite studies implicating oxidative modification of protein cysteines as a modulator of cellular processes in humans, including angiogenesis, the molecular details of the majority of these modifications, including the complete repertoire of proteins containing cysteine post-translational modifications (PTMs) and the specific sites of modification, remain largely unknown.

The mitochondrial respiratory chain is a major source of intracellular ROS generation (Storz. (2006) Sci STKE 2006, re3., herein incorporated by reference in its entirety). During aerobic respiration, superoxide (O₂⁻) can be generated at complexes I and III of the electron transport chain by partial reduction of oxygen at the mitochondrial inner membrane. The ROS are subsequently released into the mitochondrial matrix or the intermembrane space. Spontaneous or catalytic breakdown of O₂⁻ by superoxide dismutase (SOD) in the mitochondrial matrix (MnSOD) or cytoplasm (Cu/ZnSOD) results in hydrogen peroxide (H₂O₂) production. The second major source of ROS are NADPH oxidases (Rhee. (2006) Science 312, 1882-1883., herein incorporated by reference in its entirety). In particular, subunits of Nox oxidase assemble at phagosomal membranes to release millimolar quantities of H₂O₂ for phagocytic cells. Alternatively, activation of various cell surface receptors (e.g., by growth factors, cytokines, insulin, tumor necrosis factor-a) in nonphagocytic cells activates Phox oxidases that reside on the plasma membrane (Lambeth. (2004) Nat Rev Immuno 14, 181-189., herein incorporated by reference in its entirety). Phox activation results in O₂⁻ production. The O₂⁻ is rapidly dismutated to H₂O₂ that can function as an intracellular signaling molecule. Not surprisingly, several enzymes function to detoxify ROS (Stone & Yang. (2006) Antioxid Redox Signals, 243-270., herein incorporated by reference in its entirety). As mentioned above, O₂⁻ is converted to H₂O₂ by SOD enzymes. In turn, H₂O₂ is reduced by catalase, peroxidredoxins (Prx) and glutathione peroxidase (GPX). These ROS detoxifying enzymes function as antioxidants to restore redox balance under oxidative stress conditions and also downregulate ROS signaling pathways.

One of the earliest established biological functions of ROS is the generation of oxidative stress, a condition characterized by the oxidation of thiol pools, including glutathione (GSH), which can alter the redox potential of the cell (Stone & Yang. (2006) Antioxid Redox Signals, 243-270., herein incorporated by reference in its entirety). Bacteria and yeast have developed systems to sense and respond to excess ROS in their environments and higher multicellular organisms have capitalized on targeted ROS production for host defense by engulfing and killing microbes. ROS can also function as a signaling agent via preferential oxidation of a signaling protein by ROS, which either activates or inhibits its biological activity, altering its function.

For a ROS to act as a signaling molecule it should not be entirely destroyed by antioxidant enzymes. Site-specific generation of ROS by NADPH oxidases can rapidly create a bolus of ROS that is above the threshold for enzymatic reduction. In addition, the catalysts that detoxify ROS are often inactivated by ROS. The ROS signal needs to alter the activity of specific ROS-sensitive proteins, termed ROS “sensors”. The amino acid residue that is most sensitive to ROS is cysteine. In particular, a deprotonated thiol is highly susceptible to oxidation by ROS. In the majority of cytosolic proteins and non-protein thiols such as GSH, the thiols are protonated at physiological pH and are therefore, fairly unreactive toward ROS. The majority of cellular thiols react with H₂O₂ with a second order rate constant of ˜1 M⁻¹s⁻¹ (Winterbourn & Metodiewa. (1999) Free Radic Biol Med 27, 322-328., herein incorporated by reference in its entirety). In contrast, ROS “sensor” proteins typically contain cysteines that possess low pKₐs and react with H₂O₂ with second order rate constants of 10²-10⁵ M⁻¹s⁻¹ (Stone & Yang. (2006) Antioxid Redox Signals, 243-270., herein incorporated by reference in its entirety). Cysteines with low pKₐs are found in enzymes that require the thiol deprotonation as a step in their catalytic mechanisms, and in protein environments that have evolved to stabilize the deprotonated thiol. Sulfenic acids (Cys-SOH) are formed by direct nucleophilic attack of H₂O₂ by a thiolate (SEE FIG. 1). Once cellular redox balance is restored, sulfenic acids can be reduced by a thiol such as GSH. Alternatively, sulfenic acids can be further oxidized to sulfinic (CYS—SO₂H) and require energy input to reduce. Sulfonic acids (CYS—SO₃H) are formed by oxidation of sulfinic acid. At present time, there is no evidence to suggest that sulfonic acids can be reduced in a biological context and thus, these modifications may be hallmarks of oxidative stress associated with disease states (Hess et al. (2005) Nat Rev Mol Cell Biol 6,150-166., herein incorporated by reference in its entirety). Each of these oxidative cysteine modifications can have a dramatic effect on protein function, either activating or deactivating its cellular function.

Proteins that are critical for microbial response to oxidative assault were originally identified by genetic screens (Veal et al. (2007) Mol Cell 26, 1-14., herein incorporated by reference in its entirety). In bacteria, the transcription factor, OxyR can be directly oxidized in response to H₂O₂ thereby activating the transcription of antioxidant genes. When added directly to bacterial culture, OxyR rapidly senses levels of H₂O₂ as low as 5 pM. The activation of OxyR is mediated via a critical cysteine residue that becomes oxidized to Cys-SOH and in subsequent steps, results intramolecular disulfide formation and large-scale conformational rearrangements in OxyR (Zheng et al. (1998) Science 279, 1718-1721., herein incorporated by reference in its entirety). Analogous to prokaryotes, yeast also have ROS sensing systems. The transcription factor, Yapl (homologous to c-jun) plays a central role in mediating cellular responses to ROS and oxidative stress and, in response to mild H₂O₂ stress (added exogenously or as a by-product of respiratory bursts), Yapl translocates to the nucleus where it activates the transcription of antioxidant enzymes (Delaunay et al. (2002) Cell 111, 471481., herein incorporated by reference in its entirety). This response appears to be mediated by glutathione peroxidase 3 (Gpx3), an enzyme that catalyzes reduction of H₂O₂. In the current model for Yapl activation, H₂O₂ reduction converts the Gpx3 catalytic cysteine residue to Cys-SOH, and as a result of this oxidation an intermolecular disulfide-linked protein complex forms between Yapl and Gpx3. A subsequent thiol-disulfide exchange reaction leads to formation of an intramolecular disulfide bond in Yapl that masks its C-terminal nuclear export signal and results in nuclear accumulation of Yapl. In contrast to unicellular organisms that have evolved primarily to defend against oxidative stress, animals spatially and temporally channel ROS production into specific signaling pathways to achieve a desired cellular response (Rhee. (2006) Science 312, 1882-1883., Stone & Yang (2006) Antioxid Redox Signals, 243-270., herein incorporated by reference in their entireties). At the cell surface, receptor activation by numerous stimuli including growth factors, insulin and cytokines, can activate NADPH oxidase assembly and stimulate ROS production. In turn, ROS “sensor” proteins such as protein tyrosine phosphatases (PTPs) can respond to the ROS signal (Tonks. (2005) Cell 121, 667-670., herein incorporated by reference in its entirety). PTPs depend upon a deprotonated cysteine residue to catalyze dephosphorylation. Oxidation of the thiolate anion to Cys-SOH by ROS inactivates these enzymes and thus, enhances the overall level of protein tyrosine phosphorylation (SEE FIG. 2). Once redox balance is restored, the catalytic cysteine residue can be reduced by glutathione (GSH) to reactivate the phosphatase. In vitro studies also suggest that protein tyrosine phosphorylation is promoted through ROS-mediated activation of protein tyrosine kinases (PTKs) via oxidation of two critical cysteine residues (Giannoni et al. (2005) Cell Biol 25, 6391-6403., herein incorporated by reference in its entirety).

Despite numerous studies implicating oxidative modification of protein cysteines as a modulator of cellular processes, the molecular details of the majority of these modifications, including the complete repertoire of proteins containing cysteine PTMs and the specific sites of modification in vivo remain unknown due to the lack of methods to investigate these processes in living cells.

Cell migration is central to many biological and pathological processes, including heart development, tissue repair and regeneration as well as atherosclerosis and the inflammatory response. In general, cell migration can be thought of as a cyclic process (Cook-Mills, J. M. (2006) Cell Mol Biol 52,8-16., herein incorporated by reference in its entirety). The initial response of a cell to a migration signal is to polarize and extend protrusions in the direction of migration. These protrusions (e.g., lamellipodia) are driven by actin polymerization, and are stabilized by adhering to the extracellular matrix (ECM) via transmembrane receptors of the integrin family linked to the actin cytoskeleton. These adhesions serve as traction sites for migration as the cell moves forward over them, and they must be disassembled at the cell rear, allowing it to detach. Hence, cell migration integrates localized, transient signaling events with changes in cellular architecture. The initial point of contact between the lamellipodium and matrix is termed the “focal complex” which matures into stable “focal adhesions” (Nobes & Hall. (1995) Cell 51, 53-62., herein incorporated by reference in its entirety). NAPDH oxidase assembly at nascent focal complexes occurs in response to migratory stimuli. The adaptor protein TRAF4 as well as the focal contact scaffold Hic-5 mediates oxidase localization (Wu et al.(2005) J Cell Biol 171, 893-904., herein incorporated by reference in its entirety). The GTPase Rac1 is also necessary for the activation of oxidase activity. Localized production of O₂⁻ (rapidly dismutated to H₂O₂) results in a large increase in protein tyrosine phosphorylation of focal contact proteins and stimulates locomotion. Several recent reports suggest that the shift toward tyrosine phosphorylation may result from oxidative cysteine modification of the focal contact proteins PTP-PEST (inhibiting phosphatase activity) and Src and Pyk2 (activating kinase activity) (Giannoni et al. (2005) Cell Biol 25, 6391-6403., Wu et al. (2005) J Cell Biol 171, 893-904., herein incorporated by reference in their entireties). In vitro studies support the feasibility of this hypothesis: PTPs can be reversibly inactivated via cysteine oxidation (Salmeen et al. (2003) Nature 423, 769-773., herein incorporated by refeence in its entirety) and Src tyrosine kinase activity can be stimulated by oxidization of two cysteine residues (Giannoni et al. (2005) Cell Biol 25, 6391-6403., herein incorporated by refeence in its entirety). In addition, oxidant-scavenging reagents block cell motility, consistent with the requirement for ROS signals during cell migration (Wu et al.(2005) J Cell Biol 171, 893-904., herein incorporated by refeence in its entirety).

Strategies have been introduced for the enrichment of reduced cysteine and disulfide-containing peptides for proteomic analysis (Eaton. (2006) Free Radic Biol Med 40, 1889-1 899., herein incorporated by refeence in its entirety). These chemical methods exploit the chemical reactivity of cysteine's thiol (R—SH) functionality for selective capture by reacting proteins with reagents such as iodoacetamide or maleimide to covalently modify thiols. Chemical elaboration of these thiol-reactive reagents with a fluorescent or affinity tag facilitates detection and capture of modified proteins. To identify proteins that form intra- or intermolecular disulfide bonds under conditions of oxidative stress, traditional analytical techniques take advantage of the fact that oxidation to a disulfide (R—S—S—R) leads to loss of thiol groups. The loss of thiol group reactivity is then used to detect and quantify the extent of protein thiol oxidation (Leichert & Jakob. (2004) PLoS Biol 2, e333., herein incorporated by refeence in its entirety). Analogous “subtractive” approaches for sulfenic acid-containing proteins have also been reported (Saurin et al. (2004) Proc Natl Acad Sci USA 101, 17987., herein incorporated by refeence in its entirety). These methods suffer from several limitations. The most serious drawback of all previous approaches is that they are restricted to in vitro use in cell extracts. Since thiol modifications are very sensitive to cellular redox environment, methods that require cell lysis and subsequent chemical treatments cannot detect labile or short-lived cysteine PTMs characteristic of signaling events. A second limitation of these techniques is the “subtractive” nature of the analysis. Free thiols must be completely chemically blocked in order to avoid false positive identification in these assays and in practice this is very difficult to achieve. A more recently reported chemical detection method for cysteine sulfenic acids incorporates large chemical tags, such as biotin or fluorophores, into the probe (Poole et al. (2005) Bioconjug Chem 16, 1624-1628., herein incorporated by refeence in its entirety). Large chemical tags have the disadvantage of altering the specificity of protein labeling, prevent passive diffusion of the probe into cells and are often associated with high background fluorescence of unbound probe. Hence, these reagents are restricted to in vitro use. Prior to the present invention, no general chemical approaches have been developed for the identification of sulfinic- or sulfonic acid-oxidized cysteines.

### SUMMARY

Embodiments of the present invention provide compounds comprised of a reactive group (e.g. chemoselective moiety), capable of specifically reacting with a single post-translationally-modified cysteine oxidative state, connected to an azide group, by an alkyl chain. The reactive group is capable of targeting, and reacting with, a post-translationally-modified cysteine oxidative state, both in vivo and in vitro, in a protein non-specific manner. The reaction of the reactive group with a specific cysteine oxidative state results in the covalent linkage of a compound of the present invention to the post-translationally-modified cysteine oxidative state. In embodiments of the present invention, the covalent linkage of a compound of the present invention to the post-translationally-modified cysteine oxidative state results in the azide group of the present invention being displayed on the post-translationally-modified cysteine for further chemical modification.

In some embodiments, the present invention provides a compound comprised of a reactive group (e.g. chemoselective group), capable of specifically reacting with a single post-translationally-modified cysteine oxidative state, connected to an azide group, by an alkyl chain. The reactive group is capable of targeting, and reacting with, a post-translationally-modified cysteine oxidative state, both in vivo and in vitro, in a protein non-specific manner. In various aspects of the invention, the reactive group is capable of covalently and specifically binding cysteine-thiol, cysteine-sulfenic acid, cysteine-sulfinic acid, and/or cysteine-sulfonic acid, or equivalents thereof In some embodiments, the alkyl chain which links the reactive group to the azide group may be 1 to 10 carbons in length, although both longer and shorter linkers may be used.

In another aspect of the invention, the compound is cell permeable. Upon being administered, the compound is capable of efficiently entering cells. Upon having entered cells, the reactive group is capable of specifically reacting with a post-translationally-modified cysteine oxidative state. Such compounds find use in culture, in isolated tissue, and in vivo.

Embodiments of the invention provide a method for labeling a single specific post-translationally-modified cysteine oxidative state, comprising one or more steps of: a) providing a chemoselective agent capable of specifically binding a single post-translationally-modified cysteine oxidative state in a protein non-specific manner, and a sample containing the amino acid cysteine in one or more oxidative states, wherein said sample may be in vivo or in vitro, wherein said cysteines may be present in the sample as single amino acids, part of a peptide, part of a polypeptide, or part of a protein; b) administering said chemoselective agent to said sample; c) reacting of said single post-translationally-modified cysteine oxidative state with said chemoselective agent to form a chemical bond, wherein said reaction is non-specific with regards to the peptides, polypeptides, or proteins; d) administering a secondary agent to said sample; e) interacting said secondary agent with said chemoselective agent, wherein said interaction comprises covalent bonding or specific molecular recognition, wherein said interaction occurs specifically between said secondary agent and said chemoselective agent, wherein said interaction occurs non-specifically with regards to the peptides, polypeptides, or proteins; f) administering a tertiary agent to said sample; and g) forming a stable interaction between said secondary agent and said tertiary agent. In some embodiments step d occurs before step c. In some embodiments step f occurs before step c.

In some embodiments of the present invention, the first chemoselective agent is a composition of the present invention, comprised of a reactive group, capable of specifically reacting with a single post-translationally-modified cysteine oxidative state, connected to an azide group, by an alkyl chain; the reactive group is capable of targeting, and reacting with, a post-translationally-modified cysteine oxidative state, both in vivo and in vitro, in a protein non-specific manner; the reaction of the reactive group with a specific cysteine oxidative state results in the covalent linkage of a compound of the present invention to the post-translationally-modified cysteine oxidative state.

In some embodiments of the present invention, the first chemoselective agent is 3,5-dihydroxybenzaldehyde. In other embodiments of the present invention, the first chemoselective agent is para-diazonium benzaldehyde. In some embodiments of the present invention, the secondary agent is phosphene-biotin (P-Biotin). In some embodiments of the present invention, the tertiary agent is partially or wholly comprised of streptavidin. In some embodiments of the present invention, the streptavidin is conjugated to horseradish peroxidase. In some embodiments of the present invention, the enzymatic activity of said horseradish peroxidase is used to quantitatively detect said single post-translationally-modified cysteine oxidative state. In some embodiments of the present invention, streptavidin is part of a separation medium, wherein said separation medium is used to purify amino acids, peptides, polypeptides, and proteins through the affinity of streptavidin for biotin. In some embodiments of the present invention, separation medium is affinity chromatography. In some embodiments of the present invention, the first chemoselective agent is 5,5-dimethyl-1,3-cyclohexadione (Dimedone). In some embodiments of the present invention, the secondary agent is an antibody capable of specific molecular recognition of Dimedone, wherein a stable interaction is formed between said antibody and said Dimedone upon said specific molecular recognition.

In some embodiments, the present invention provides a composition comprising a compound of Formula I:

C-L-H

wherein C is a chemoselective moiety configured to specifically bind a single post-translationally-modified cysteine oxidative state, selected from cysteine-thiol, cysteine-sulfenic acid, cysteine-sulfinic acid, and cysteine-sulfonic acid, wherein the binding is capable of occurring in vivo and in vitro, and wherein the binding occurs in a context independent manner (e.g. free cysteine, cysteine which is part of a protein, etc.); wherein L is a linker moiety; and wherein H is a handle moiety. In some embodiments, the handle moiety comprises an azide group. In some embodiments, the compound is cell permeable. In some embodiments, the chemoselective moiety covalently and specifically binds cysteine-sulfenic acid. In some embodiments, the chemoselective moiety covalently and specifically binds cysteine-sulfinic acid. In some embodiments, the chemoselective moiety covalently and specifically binds cysteine-thiol. In some embodiments, the chemoselective moiety comprises 3,5-dihydroxybenzaldehyde. In some embodiments, the chemoselective moiety comprises para-diazonium benzaldehyde. In some embodiments, the chemoselective moiety comprises a diazonium-containing compound. In some embodiments, the handle comprises a ligand, antigen, or reactive group for a secondary binding agent.

In some embodiments, the present invention provides a method comprising: a) providing: i) a chemoselective agent capable of specifically binding a single post-translationally-modified cysteine oxidative state in a context independent manner, and ii) a sample containing the amino acid cysteine in one or more oxidative states, wherein the sample may be in vivo or in vitro, wherein the cysteines may be present in the sample as single amino acids, part of a peptide, part of a polypeptide, or part of a protein; b) administering the chemoselective agent to the sample; and c) reacting of the single post-translationally-modified cysteine-oxidative state with the chemoselective agent to form a chemical bond, wherein the reaction is non-specific with regard to the peptides, polypeptides, or proteins. In some embodiments, the present invention further comprises: d) providing a secondary agent, wherein said secondary agent is configured to bind to the chemoselective agent; e) administering said secondary agent to said sample; and f) interacting said secondary agent with said chemoselective agent, wherein said interaction comprises covalent bonding or specific molecular recognition, wherein said interaction occurs specifically between said secondary agent and said chemoselective agent, wherein said interaction occurs non-specifically with regards to the peptides, polypeptides, or proteins. In some embodiments, the present invention further comprises: g) providing a tertiary agent; h) administering the tertiary agent to the sample; and i) forming a stable interaction between the secondary agent and the tertiary agent. In some embodiments, the chemoselective agent comprises an azido handle. In some embodiments, the secondary agent comprises phosphene-biotin (P-Biotin). In some embodiments, the tertiary agent comprises streptavidin. In some embodiments, the streptavidin is conjugated to horseradish peroxidase. In some embodiments, the chemoselective agent comprises dimedone. In some embodiments, the the secondary agent is an antibody, wherein the antibody is capable of specific molecular recognition of dimedone, wherein a stable interaction is formed between the antibody and dimedone upon specific molecular recognition. In some embodiments, the single post-translationally-modified cysteine oxidative state comprises a thiol, sulfenic acid, sulfinic acid, or sulfonic acid.

### DESCRIPTION OF FIGURES

The foregoing summary and detailed description is better understood when read in conjunction with the accompanying drawings which are included by way of example and not by way of limitation.

FIG. 1 shows a schematic illustrating the higher oxidation states of cysteine.

FIG. 2 shows a schematic illustrating the in vivo labeling of cysteine sulfenic acid with probe (1).

FIG. 3 shows the chemical structures exemplary sulfenic acid-specific probes: a) azido-cysteine sulfenic acid probes (1-3), b) cysteine sulfenic acid-specific chemoselective moieties (4-5).

FIG. 4 shows a) the reaction of sulfinic acid with aryl diazonium; b) the chemical structure of azido-cysteine sulfinic acid probe (6); and c) sulfinic acid-specific chemoselective moiety (7), X=functional group for detection and Y=electron withdrawing functional group such as —F or —OCH3.

FIG. 5 shows an exemplary thiol-specific chemoselective moiety (8); X=halogen or CN.

FIG. 6 shows a schematic illustrating the reaction of cysteine sulfenic acid, labeled with probe (1), with P-biotin to yield specifically biotinylated protein.

FIG. 7 shows the evaluation of probe (1) reactivity with the cysteine protease papain using: a) kinetic analysis, b) streptavidin-HRP Western blot, and c) mass spectrometry (top: reduced papain treated with (1), bottom: oxidized papain treated with (1)).

FIG. 8 shows a polyacrylimide gel demonstrating the in vivo detection of sulfenic acid-containing proteins (lane 1: absence of (1), lane 2: presence of (1)).

FIG. 9 shows a schematic illustrating the synthesis of a Dimedone thioether hapten and its conjugation to a carrier protein: a) attachment of an Fmoc handle to bromonated Dimedone via a thioether linkage, b) conversion of Fmoc to a primary amine, c) synthesis and attachment of mercaptolinker, and d) conjugation of thioether hapten with mercaptolinker to carrier protein.

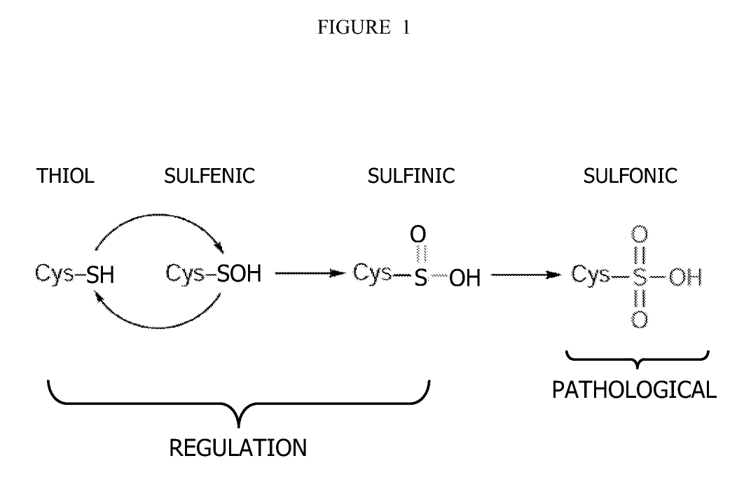

### DETAILED DESCRIPTION

Embodiments of the present invention provide cell-permeable chemical probes that selectively recognize particular cysteine PTMs, allowing for identification of the relevant oxidation targets and the dissection of complex ROS signaling networks in vivo as well as in vitro. For example, embodiments of the present invention provide bioorthogonal chemical probes with the ability to identify proteins that are modified by oxidative cysteine PTMs in living cells. Identification of ROS protein sensors and elucidating their mechanisms of activation by the compositions and methods herein will advance knowledge of ROS-mediated signaling and find use in the identification of new therapeutic targets. In some embodiments, the present invention utilizes the unique chemical reactivity of higher cysteine oxidation states to identify ROS “sensor” proteins. In some embodiments, the chemical probes and methods of the current invention are used to examine when PTPs and PTKs become oxidatively modified in human endothelial cells in response to migratory stimuli.

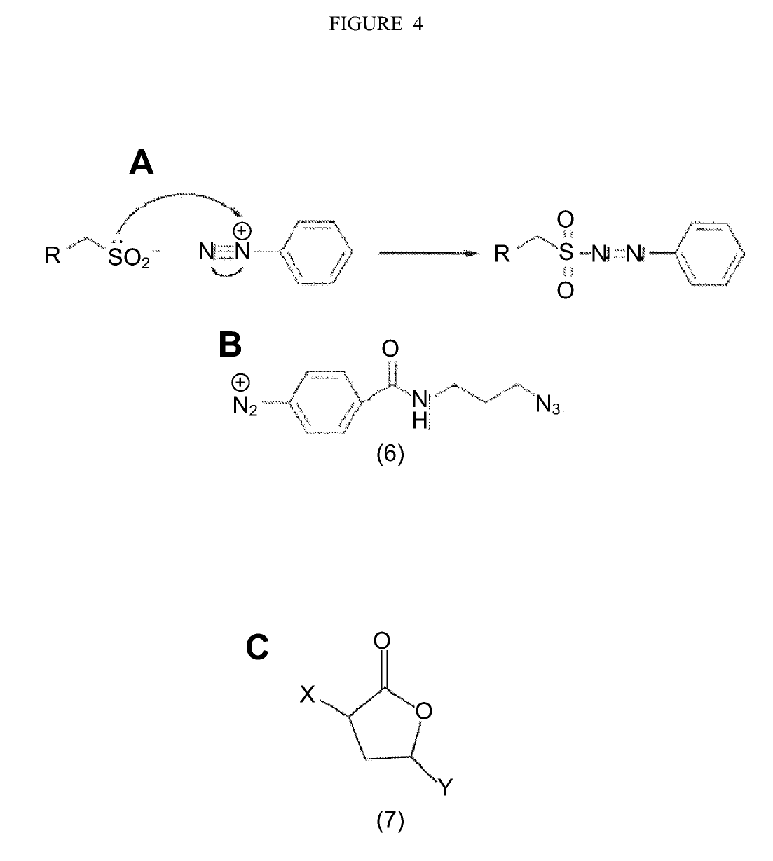

Some embodiments of the present invention positively provide specific cysteine modifications, as opposed to relying on subtractive analysis, and facilitate investigation of ROS signaling in real time, which is not possible with chemical manipulation using cell extracts. In a some embodiments, chemical probes provided by the present invention contain two features (see FIG. 4A): a reactive group (e.g. chemoselective moiety) that covalently modifies a specific oxidation state (e.g., Cys-SOH, Cys-SO₂H, etc.), and a azide chemical functionality (R—N₃) which functions as a chemical handle, and permits selective coupling with phosphine reagents via the bioorthogonal Staudinger ligation or alkynes via Huisgen [3+2] cycloaddition for detection and isolation by affinity purification for subsequent mass spectrometry (MS) analysis (SEE FIG. 4-9) (Agard et al. (2006) ACS Chem Biol 1, 644-648., herein incorporated by reference in its entirety). In other embodiments, chemical probes which bind to specific cysteine modifications are recognizable by antibodies. In some embodiments of the invention, these antibodies are used to characterize, quantify, or purify proteins with specific cysteine modifications. In some embodiments, the present invention's multi-step labeling approach effectively decouples the in vivo protein-labeling step from subsequent visualization and proteomic analysis.

In some embodiments, the present invention provides compositions and methods for the characterization (e.g. identification, purification, isolation, analysis, etc.) of cysteine oxidative states. In some embodiments, compositions and methods of the present invention are selective for a single oxidative state of cysteine (e.g. thiol, sulfenic acid, sulfinic acid, sulfonic acid, etc.). In some embodiments, the present invention provides one or more agents (e.g. 1 agent, 2 agents, 3 agents, 4 agents, 5 agents, 10 agents, etc.) for characterizing post-translationally modified cysteines (e.g. thiol, sulfenic acid, sulfinic acid, sulfonic acid, etc.). In some embodiments, a chemoselective agent and secondary agent are provided for the characterization of post-translationally modified cysteines. In some embodiments, a chemoselective agent, secondary agent, and tertiary agent are provided for the characterization of post-translationally modified cysteines.

In some embodiments, the present invention provides a chemoselective agent configured for recognition of a single cysteine oxidative state (e.g. thiol, sulfenic acid, sulfinic acid, sulfonic acid, etc.). In some embodiments, a chemoselective agent is a first agent in a multistep method of characterization, identification, purification, isolation, and/or analysis of a cysteine oxidative state. In some embodiments, a chemoselective agent coordinates, covalently binds, non-covalently binds, recognizes, or interacts with a single cysteine oxidative state (e.g. thiol, sulfenic acid, sulfinic acid, sulfonic acid, etc.). In some embodiments, a chemoselective agent covalently binds a single cysteine oxidative state (e.g. thiol, sulfenic acid, sulfinic acid, sulfonic acid, etc.). In some embodiments, a chemoselective agent binds or interacts with cysteine in a context independent manner (e.g. free cysteines; cysteines that are part of a peptide, polypeptide, or protein). In some embodiments, following reaction with an oxidized cysteine, a chemoselective agent of the present invention provides a tag, label, handle, etc. for further steps of identification, characterization, purification, isolation, analysis, etc. In some embodiments, by binding to a post-translationally modified cysteine, a chemoselective agent converts the cysteine into a characterizable group (e.g. by addition of a handle, tag, or label onto the cysteine). In some embodiments, a chemoselective agent provides a cysteine-reactive moiety (e.g. cysteine sulfenic acid reactive, cysteine sulfinic acid reactive, cysteine sulfonic acid reactive, etc.) and a second moiety (e.g. tag moiety, handle moiety, label moiety, ligand moiety etc). In some embodiments, the cysteine-reactive moiety and the second moiety are connected by a linker moiety.

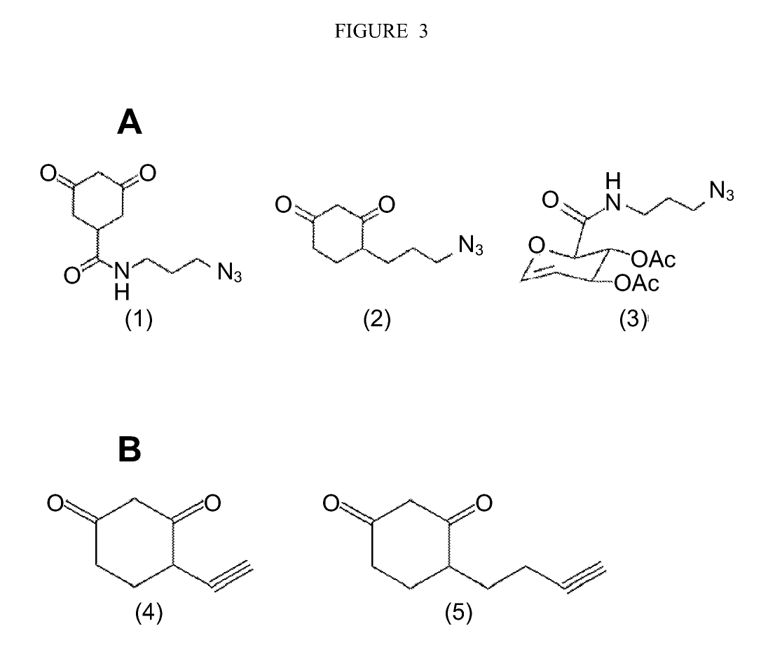

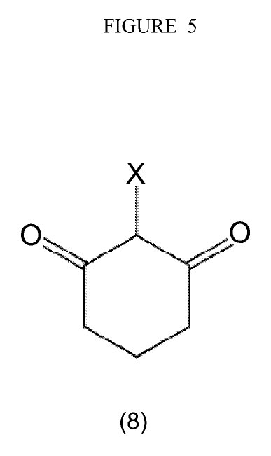

In some embodiments, a chemoselective agent comprises a chemoselective moiety. In some embodiments, a chemoselective moiety comprises a cysteine-reactive moiety. In some embodiments, a chemoselective agent comprises a cysteine-reactive moiety and one or more other moieties (e.g. handle moiety, linker moiety, label moiety, etc.). In some embodiments, a cysteine-reactive moiety is provides selectivity for a single cysteine oxidative state (e.g. thiol, sulfenic acid, sulfinic acid, sulfonic acid, etc.). In some embodiments, a chemoselective agent comprises a cysteine-reactive moiety selected from thiol-selective moity, sulfenic acid-selective moiety, sulfinic acid-selective moiety, sulfonic acid-selective moiety, etc. In some embodiments, a cysteine-reactive moiety provides the chemoselectivity of a chemoselective agent of the present invention. In some embodiments, chemoselective agents provide a modular structure in which one or more of the moieties (e.g. chemoselective moiety, linker moiety, handle moiety, etc.) can be substituted to yield a chemoselective agent with varied properties. In some embodiments, a cysteine sulfenic acid-selective moiety comprises a 1,3-cyclohexadione-related compound, such as probe 1 (SEE FIG. 3), probe 2 (SEE FIG. 3), other cyclic diones such as probe 4 and probe 5 (SEE FIG. 3), derivates thereof, or related compounds. In some embodiments, a cysteine sulfenic acid-selective moiety comprises a 3,4-dihydro-2H-pyran-related compound, such as probe 3 (SEE FIG. 3), derivatives thereof, or other similar or related compound. In some embodiments, a cysteine sulfinic acid-selective moiety comprises a diazonium-containing compound or diazonium-related compound, such as probe 6 (SEE FIG. 4), derivatives thereof or related compounds. In some embodiments, a cysteine sulfinic acid-selective moiety comprises a compound, such as probe 7 (SEE FIG. 4), which comprises functional group for detection and an electron withdrawing functional group such as —F or —OCH₃. In some embodiments, a cysteine sulfonic acid-selective moiety comprises a compound configured to selectively react with cysteine sulfonic acid over other oxidative states of cysteine. In some embodiments, a cysteine sulfonic acid-selective moiety comprises a compound configured to selectively react with cysteine sulfonic acid over other functional groups, reactive groups, amino acids, modified amino acids, etc. In some embodiments, a cysteine thiol-selective moiety comprises a compound such as probe 8 (SEE FIG. 5) which contains a halogen or cyano group. In some embodiments, a cysteine thiol-selective moiety comprises a compound configured to selectively react with cysteine sulfonic acid over other oxidative states of cysteine. In some embodiments, a cysteine thiol-selective moiety comprises a compound configured to selectively react with cysteine sulfonic acid over other functional groups, reactive groups, amino acids, modified amino acids, etc.

In some embodiments, a chemoselective agent comprises a second functional moiety in addition to a cysteine-reactive moiety. In some embodiments, a chemoselective agent comprises a handle moiety. In some embodiments, a handle moiety is configured to provide a functional group for additional manipulation or characterization of a cysteine residue, following reaction with the chemoselective agent. In some embodiments, a handle moiety is a tag configured to provide a functional group for additional manipulation or characterization. In some embodiments, a handle moiety is a hapten moiety configured to render cysteine residues recognizable by an antibody. In some embodiments, a handle moiety is a label configured to provide a detectable group (e.g. fluorescent, radiolabel, contrast agent, etc.) for additional characterization, localization, or identification of cysteine residues. In some embodiments, a handle moiety is a ligand moiety configured to provide a functional group capable of reaction or coordination with an additional chemical entity (e.g. small molecule, protein, label, tag, antibody, etc.). In some embodiments, a functional group on a chemoselective agent may be capable of functioning as more than one of a handle, tag, hapten, label, ligand, etc. In some embodiments, a handle moiety comprises an azide group. In some embodiments, an azide group is covalently attached to a cysteine-reactive moiety (chemoselective moiety). In some embodiments, an azide group and a cysteine-reactive moiety are covalently attached by a linker moiety. In some embodiments, an azide group is configured to provide a site of additional modification of cysteine residue following reaction with a azide-containing chemoselective agent. In some embodiments, an azide group reacts specifically with P-biotin. In some embodiments, an azide group reacts specifically with P-biotin to form a covalent bond. In some embodiments, a handle comprises a functional group or moiety which is configured to specifically react with a secondary agent, thereby providing a site for additional modification, manipulation or characterization. In some embodiments, a handle moiety comprises 5,5-dimethyl-1,3-cyclohexadione (dimedone), a dimedone group, a dimedone thioether, dimedone-related compounds, or derivatives thereof. In some embodiments, a dimedone handle is configured to provide a site of antibody recognition. In some embodiments, a dimedone handle is configured to provide a site of further modification or manipulation. In some embodiments, a handle moiety may comprise any functional group configured to add a tag, handle, label, hapten, and/or ligand functionality onto a chemoselective agent.

In some embodiments, the present invention provides a secondary agent configured to recognize a first agent or chemoselective agent. In some embodiments, the present invention provides a secondary agent configured to recognize a first agent or chemoselective agent which is bound to a cysteine residue. In some embodiments, a secondary agent coordinates, covalently binds, non-covalently binds, recognizes, or interacts with a chemoselective agent bound to a cysteine and/or an unbound chemoselective agent. In some embodiments, a secondary agent interacts with the handle moiety of a first chemoselective agent. In some embodiments, a secondary agent is chemoselective. In some embodiments, a secondary agent interacts specifically or specifically binds to a region of a chemoselective agent (e.g. a handle moiety). In some embodiments, a secondary agent comprises an antibody (e.g. polyclonal antibody, monoclonal antibody, humanized antibody, fusion antibody, etc.). In some embodiments, a secondary agent comprises and antibody which recognizes a region of a chemoselective agent as an antigen. In some embodiments, an antibody is capable of recognizing and binding to a chemoselective agent which is bound to a cysteine residue. In some embodiments, binding of an antibody to a chemoselective agent which is bound to a cysteine residue provides a method of characterization, identification, purification, isolation, and/or analysis of the cysteine residue or the protein, polypeptide, or peptide of which the cysteine is part. In some embodiments, the present invention provides antibodies configured to bind to any chemoselective agents described herein. In some embodiments, the present invention provides antibodies configured to bind to any handle moieties (e.g. ligand moieties described herein. In some embodiments, a secondary agent is a small molecule (e.g. P-biotin), macromolecule (e.g. antibody, protein, nucleic acid, etc.), molecular complex, or compound capable of covalently or non-covalently binding to a chemoselective agent of the present invention (e.g. binding to a handle moiety of a chemoselective agent). In some embodiments, a small molecule secondary agent binds specifically to a handle moiety of a chemoselective agent. In some embodiments, binding of a secondary agent provides additional or altered functionality to a chemoselective agent for characterization of cysteine oxidative states (e.g. imaging functionality, purification functionality, etc.). In some embodiments, a secondary agent binds to a specific group on a cysteine-bound chemoselective agent without regard for the cysteine oxidative state. In some embodiments, example pairs of chemoselective agents and secondary agents include: an azide containing chemoselective agent and a P-biotin containing secondary agent, a dimedone-containing chemoselective agent and a dimedone antibody secondary agent, a biotin-containing chemoselective agent and a streptavidin-containing secondary agent, etc. In some embodiments, a secondary agent comprises a binding moiety which coordinates or covalently binds the handle moiety of a chemoselective agent, and further comprises a secondary handle moiety (e.g. tag, ligand, label, hapten, etc.). In some embodiments, the binding moiety and secondary handle moiety of a secondary agent are directly attached. In some embodiments, the binding moiety and secondary handle moiety of a secondary agent are connected by a linker moiety.

In some embodiments, the present invention provides a tertiary agent configured to recognize a secondary agent. In some embodiments, a tertiary agent is configured coordinate or bind (e.g. covalently or non-covalently) the secondary handle of a secondary agent. In some embodiments, the present invention provides a tertiary agent configured to recognize a secondary agent which is bound to a chemoselective agent. In some embodiments, the present invention provides a tertiary agent configured to recognize a secondary agent which is bound to a chemoselective agent bound to a cysteine residue. In some embodiments, a tertiary agent coordinates, covalently binds, non-covalently binds, recognizes, or interacts with a secondary agent bound to a chemoselective agent or an unbound secondary agent. In some embodiments, a tertiary agent interacts with the secondary handle moiety of a secondary agent. In some embodiments, a tertiary agent is chemoselective. In some embodiments, a tertiary agent interacts specifically or specifically binds to a region of a secondary agent (e.g. a secondary handle moiety). In some embodiments, a tertiary agent comprises an antibody (e.g. polyclonal antibody, monoclonal antibody, humanized antibody, fusion antibody, etc.). In some embodiments, a tertiary agent comprises and antibody which recognizes a region of a secondary agent as an antigen. In some embodiments, an antibody is capable of recognizing and binding to a secondary agent which is bound to a chemoselective agent. In some embodiments, binding of an antibody to a secondary agent, which is bound to a chemoselective agent, which is in turn bound to a cysteine residue, provides a method of characterization, identification, purification, isolation, and/or analysis of the cysteine residue or the protein, polypeptide, or peptide of which the cysteine is part. In some embodiments, the present invention provides antibodies configured to bind to any secondary agents, as defined above. In some embodiments, the present invention provides antibodies configured to bind to any secondary handle moieties (e.g. ligand moieties), as defined above. In some embodiments, a secondary agent is a small molecule, macromolecule (e.g. antibody, protein (e.g. streptavidin), nucleic acid, etc.), molecular complex, or compound capable of covalently or non-covalently binding to a secondary agent of the present invention (e.g. binding to a secondary handle moiety of a secondary agent). In some embodiments, a small molecule tertiary agent binds specifically to a secondary handle moiety of a secondary agent. In some embodiments, a tertiary agent binds to a specific group on a secondary agent without regard for the cysteine oxidative state of an upstream cysteine. In some embodiments, examples of pairs of secondary agents and tertiary agents include: dimedone-containing secondary agent and dimedone antibody tertiary agent, a biotin-containing secondary agent and a streptavidin-containing tertiary agent, etc. In some embodiments, a tertiary agent comprises a binding moiety which coordinates or covalently binds the secondary handle moiety of a secondary agent, and further comprises a method of purifying the resulting complex (e.g. cysteine containing peptide or protein, chemoselective agent, secondary agent, and tertiary agent).

In some embodiments, an agent of the present invention (e.g. chemoselective agent, secondary agent, tertiary agent, etc.) of the present invention comprises a linker moiety. The present invention is not limited to any particular linker moiety. In some embodiments, the linker connects two moieties (e.g. chemoselective moiety and handle moiety). In some embodiments, the linker region covalently connects two moieties In some embodiments, a chemoselective moiety is connected to more than one handle moiety by multiple linkers. In some embodiments, linkers may be enzyme cleavable, such that exposure to specified enzyme cleaves the linker and separates the connected moieties. In some embodiments, the linker is a single covalent bond, or a covalent linkage that is linear or branched, cyclic or heterocyclic, saturated or unsaturated. In some embodiments, the linker comprises 1-50 non-hydrogen atoms (in addition to hydrogen atoms) selected from the group of C, N, P, O and S (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, or 50 non-hydrogen atoms). In some embodiments, the linker comprises 1-40 non-hydrogen atoms (in addition to hydrogen atoms). In some embodiments, the linker comprises 1-30 non-hydrogen atoms (in addition to hydrogen atoms). In some embodiments, the linker comprises 1-20 non-hydrogen atoms (in addition to hydrogen atoms). In some embodiments, the linker comprises 1-10 non-hydrogen atoms (in addition to hydrogen atoms). In some embodiments, the linker comprises 1-5 non-hydrogen atoms (in addition to hydrogen atoms).In some embodiments, the linker comprises 5-10 non-hydrogen atoms (in addition to hydrogen atoms). In some embodiments, the linker comprises any combination of alkyl, ether, thioether, polyether, amine, alkyl amide, ester, carboxamide, sulfonamide, hydrazide bonds and aromatic or heteroaromatic bonds. In some embodiments, the linker comprises a polymer (e.g. nucleic acid, polypeptide, lipid, or polysaccharide), a peptide linker, a modified peptide linker, a Poly(ethylene glycol) (PEG) linker, a streptavidin-biotin or avidin-biotin linker, polyaminoacids (eg. polylysine), functionalised PEG, polysaccharides, glycosaminoglycans, dendritic polymers such as described in WO93/06868 and by Tomalia et al. in Angew. Chem. Int. Ed. Engl. 29:138-175 (1990), PEG-chelant polymers such as described in W94/08629, WO94/09056 and WO96/26754, oligonucleotide linker, phospholipid derivatives, alkenyl chains, alkynyl chains, disulfide, or a combination thereof. In some embodiments, a linker moiety comprises any covalent molecular connector capable of stringing together a first and second moiety (e.g. chemoselective moiety and handle moiety). One of ordinary skill in the art will further appreciate that the above linkers are not intended to be limiting.

The present invention further provides methods of using the above compositions for research, diagnostic, drug screening, and therapeutic purposes. In some embodiments, compounds and methods of the present invention find utility in the characterization (e.g. isolation, purification, identification, labeling, etc.) of cysteine oxidative states (e.g. thiol, sulfenic acid, sulfinic acid, sulfonic acid, etc.). In some embodiments, one or more compounds of the present invention (e.g. chemoselective agents, secondary agent, tertiary agent, etc.) bind to a one or more cysteine residues. In some embodiments, the cysteine-bound compounds (e.g. chemoselective agent and secondary and/or tertiary agents) provide a handle or tag for purifying (e.g. affinity purifying, etc.) cysteines of a selected oxidative state (e.g. free cysteine, cysteine within a peptide or protein, cysteine-thiol, cysteine-sulfinic acid, cysteine-sulfeneic acid, cysteince sulfonic acid, etc.). In some embodiments, cysteines bound by compounds of the present invention are subjected to purification techniques which are well known to those of skill in the art (e.g. column purification, gel electrophoresis, precipitation, etc.). In some embodiments, the cysteine-bound compounds (e.g. chemoselective agent and secondary and/or tertiary agents) provide a label (e.g. flurescent label, radiolabel, contrast agent) or tag (e.g. biotin) for identifying or characterizing the presence or location of cysteines of a selected oxidative state (e.g. free cysteine, cysteine within a peptide or protein, cysteine-thiol, cysteine-sulfinic acid, cysteine-sulfeneic acid, cysteince sulfonic acid, etc.).

As used herein a “sample” refers to anything capable of being subjected to the compositions and methods provided herein. In some embodiments, the sample comprises or is suspected to comprise one or more cysteine amino acids comprising an oxidative state selected from thiol, sulfenic acid, sulfinic acid, and sulfonic acid. The sample may be in vitro of in vivo. In some embodiments, the samples are “mixture” samples, which samples from more than one subject or individual. In some embodiments, the methods provided herein comprise purifying the sample. In some embodiments, the sample is purified or unpurified protein. In some embodiments, a sample may be from a clinical or research setting. In some embodiments, a sample may comprise cells, fluids (e.g. blood, urine, cytoplasm, etc.), tissues, organs, lysed cells, etc. In some embodiments, a sample may be derived from a subject. As used herein, the term “subject” refers to any animal including, but not limited to, insects, humans, non-human primates, vertebrates, bovines, equines, felines, canines, pigs, rodents, and the like.

### EXPERIMENTAL

The following examples are illustrative, but not limiting, of the methods and compositions of the present invention. Other suitable modifications and adaptations of the variety of conditions and parameters normally encountered those skilled in the art are within the spirit and scope of the invention.

### Example 1

### Compositions and Methods to Synthesize Chemical Probes Targeting Post-Translationally Modified Cysteine Oxidative States

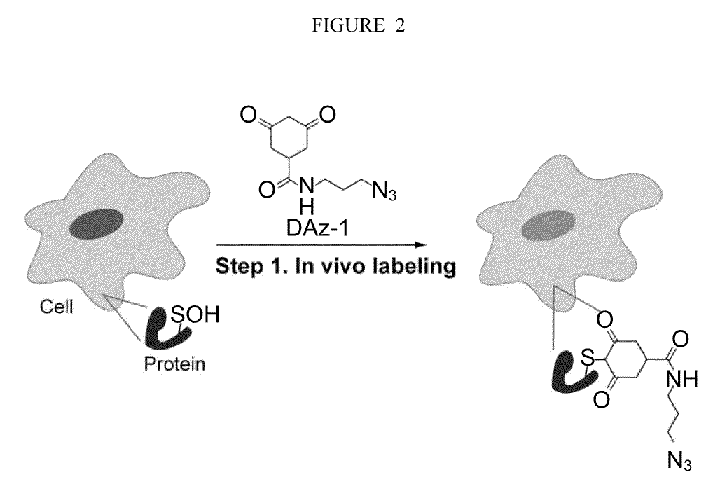

This example describes specifically targeting cysteine sulfenic acids with a chemical probe using a 1,3-cyclohexadione as a chemical scaffold. This compound reacts specifically with cysteine sulfenic acids to form a stable thiol ether linkage (SEE FIG. 2) (Benitez & Allison. (1974) J Biol Chem 249, 6234-6243., herein incorporated by reference in its entirety). The selectively of this small molecule for cysteine sulfenic acids in proteins has been demonstrated, making it an ideal for probe design (Saurin et al. (2004) Proc Natl Acad Sci USA 101,17982-17987., Poole et al. (2005) Bioconjug Chem 16, 1624-1628., Reynaert et al. (2006) Proc Natl Acad Sci USA 103, 13086-13091., herein incorporated by reference in their entireties). Probe (1) (see FIG. 3) was synthesized starting from 3,5-dihydroxybenzoic acid over five steps in 47% yield (m/z 239.1 M⁺+H⁺). The 1,3-cyciohexadione analog (2) (m/z 218.11 M⁺+Na⁺), was synthesized via LOA-mediated alkylation of 1,3-cyclohexadione at C2 (SEE FIG. 3). Sulfenic acids have also been demonstrated to add across activated alkenes such as 3,4-Oihydro-2Hpyran (Benitez & Allison. (1974) J Biol Chem 249, 6234-6243., Claiborne et al. (1999) Biochemistry 38, 15407-15416., herein incorporated by reference in their entireties). In some embodiments, the present invention uses the reactivity of this scaffold in the context of probe (3) as an alternative to the dione probes (SEE FIG. 3).

Until recently, hyperoxidized cysteine residues (CYS—SO₂H or CYS—SO₃H) were considered to be irreversible modifications (Hamann et al. (2002) Methods Enzymol 348, 146-156., herein incorporated by reference in its entirety). The recent discovery of sulfiredoxin in yeast (with homologs in humans) has challenged this paradigm and suggests a role for these functionalities in sensing changes in the cellular redox environment (Biteau et al. (2003) Nature 425, 980-984., herein incorporated by reference in its entirety). In addition to its functional significance in Prxs, oxidation of cysteine to sulfinic acid has been implicated in the activation of matrix metalloproteases (MMPs) and nitrile hydrase by modulating protein metal-binding properties (Fu et al. (2001) J Biol Chem 276, 41279-41287., Murakami et al. (2000) Protein Sci 9, 1024-1030., herein incorporated by reference in their entireties). Sulfinic acids react with soft electrophiles, such as diazonium salts (SEE FIG. 4A) (Ritchie et al. (1961) Journal of the American Chemical Society 83,4601-4605., herein incorporated by reference in its entirety). In some embodiments, the present invention provides sulfinic acid-modified proteins via azido-aryl diazonium probes (6) synthesized from p-nitrobenzoic acid starting material (SEE FIG. 4B). Although, tyrosine residues can also react slowly with diazonium salts at basic pH (e.g., pH>9) (Schlick et al. (2005) J Am Chem Soc 127, 3718-3723., herein incorporated by reference in its entirety), this side reaction is avoided by carrying out our reactions under neutral pH conditions (sulfinic acid has a low pKₐ ˜2). At pH 7.4, no reaction between tyrosine (pKₐ 9.7) and aryl diazonium was detected, whereas the 2nd order rate constant for reaction of benzene sulfinic acid at this pH is >103 M⁻¹ S⁻¹. Diazonium probes have also been shown to be biocompatible with protein samples (Schlick et al. (2005) J Am Chem Soc 127, 3718-3723., herein incorporated by reference in its entirety). In addition to its utility in vivo, in some embodiments, the present invention provides the only reagent available to probe cysteine sulfinic acid in vitro.

### Example 2

### Characterization of Post-Translationally-Modified Cysteine Probe Reactivity

Proteins that are covalently modified by the azido-label can be detected via phosphine-PEGbiotin (P-Biotin) reagent (SEE FIG. 5). The chemoselective reaction that takes place between the azide and the phosphine moieties (known as the Staudinger ligation) yields a stable amide bond between the azide-modified protein (protein-N3) and the biotin affinity handle (Saxon & Bertozzi. (2000) Science 287, 2007-2010., herein incorporated by reference in its entirety). The P-Biotin reagent has been used to detect and isolate proteins modified with by an azide functional group in numerous functional and proteomic studies (Agard et al. (2006) ACS Chem Biol 1, 644-648., Hang et al. (2007) J Am Chem Soc 129, 2744-2745., Hang et al. (2006) ACS Chem Biol 1, 713-723., herein incorporated by reference in their entirety). We have synthesized gram quantities of this compound in good yield over six steps (m/z 793.4 M⁺+H⁺). Capitalizing on the high-affinity interaction between the streptavidin protein and biotin, proteins that have been modified by P-Biotin can be detected in a western blot format using streptavidin-HRP or captured on solid phase using commercially available streptavidin-coated beads. For proteomic purposes, immobilization on solid phase allows for stringent washing to remove unmodified proteins. Isolated proteins can be trypsinized and the resulting peptides analyzed by tandem MS-MS in order to establish the identity of the proteins giving rise to the labeled peptides and the sites of thiol modification.

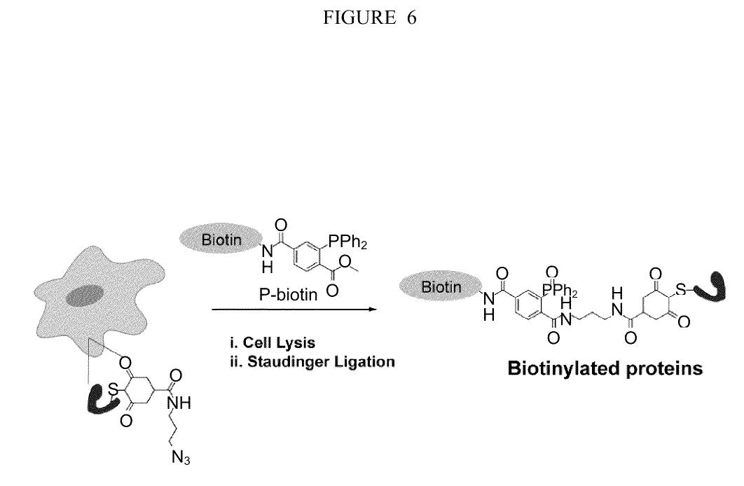

The cysteine protease, papain is a commercially available protein containing a catalytic cysteine that readily undergoes oxidizes to sulfenic and sulfinic acid. This protein contains seven cysteines, six of which can participate in disulfide bonds. The seventh cysteine is the catalytic nucleophile and due to its low pKa and unique protein environment, the thiol is highly susceptible to oxidation to sulfenic and sulfinic acid. As reported in the literature and confirmed in experiments conducted during the development of the present invention, both oxidative modifications completely inactivate papain activity. In the case of sulfenic acid formation however, activity can be restored by reduction with a small thiol such as dithiothreitol (DTT). Papain was used to test the selectivity of probe (1) for protein sulfenic acids in kinetic, western blot, and MS experiments (SEE FIG. 6). Papain activity was monitored via cleavage of a colorimetric substrate, BAPNA (SEE FIG. 6A). Treatment of papain with one equivalent of H₂O₂ oxidized the catalytic cysteine and inhibited its activity. The inhibition could be reversed through the action of the reducing agent dithiothreitol (DTT), as expected since sulfenic acids can be reduced by thiols. The existence of the cysteine sulfenic acid was secondarily confirmed by the reagent NBD-Cl, which forms a conjugate with protein sulfenic acids that can be detected by an absorption maximum at 350 nm. In contrast, reaction of oxidized papain with (1) resulted in complete loss of activity that could not be restored by DTT. This result is due to the thioether linkage formed at the catalytic cysteine with probe (1). Incubation of active papain with a large excess of (1) had no effect on catalytic activity, since (1) does not react with thiols. We have also used (1) in combination with the P-biotin detection reagent (SEE FIG. 5) to selectively detect sulfenic acid-modified papain by western blot analysis (SEE FIG. 6B). Treatment of papain with a single equivalent of peroxide resulted in the covalent modification of papain sulfenic acid by probe (1) and a robust western blot signal. In the absence of oxidant, a faint signal was observed that corresponded to reaction of probe (1) with a small fraction of papain sulfenic acid that existed in the protein preparation prior to peroxide stimulation, as confirmed by detection with NBD-CI. The specific addition of (1) to papain sulfenic acid was directly confirmed by ESI-MS analysis. No adduct was observed between (1) and papain in the thiol form (SEE FIG. 6C). In contrast, papain treated with one equivalent of H₂O₂ was robustly labeled by (1). The small percentage of papain sulfinic acid that resulted from H₂O₂ treatment did not react with (1), further demonstrating the specificity of this reagent.

### Example 3

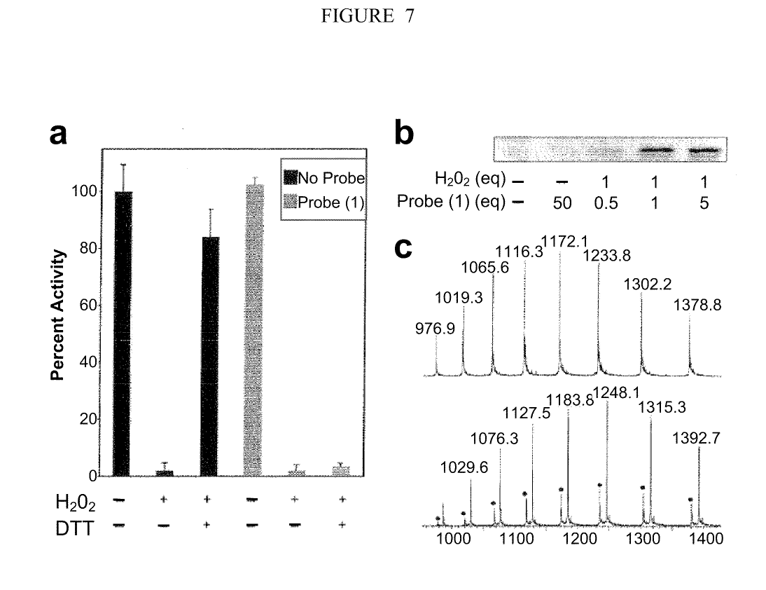

Evaluation of Cell Permeability and Toxicity in Living Cells To analyze oxidative cysteine PTMs in a living cell the azido-probes should be cell permeable and nontoxic. In order to evaluate cell permeability and toxicity of the probes in living cells, the effect of probe (1) on the viability of HUVEC, Jurkat, and Ramos B-cell lines using propidium iodide staining and flow cytometry analysis was assessed. In these experiments, no adverse effects on cell viability or changes in cell morphology were observed after 1 hr incubation with probe (1). To assess cell permeability, cell lysates were prepared for 1 hr in the presence or absence of probe (1). Cells were washed with PBS prior to harvest to remove any probe remaining in media. Robust protein labeling was in the presence of probe (1) while only minor signal from endogenously biotinylated proteins was observed in the absence of probe (SEE FIG. 7). Furthermore, no effect on the growth rate of E. coli or yeast was observed in the presence of probe (1). These studies indicate that that the azido-probe is capable of entering cells, and exhibit no toxicity towards the cells.

### Example 4

### Compositions and Methods to Generate Antibodies against Post-Translationally-Modified Cysteine Oxidative States

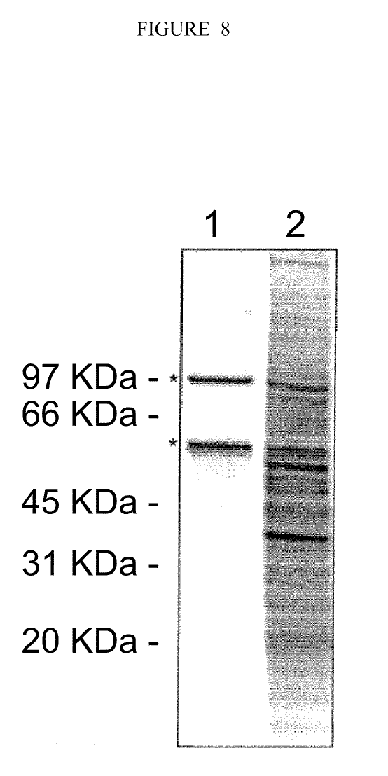

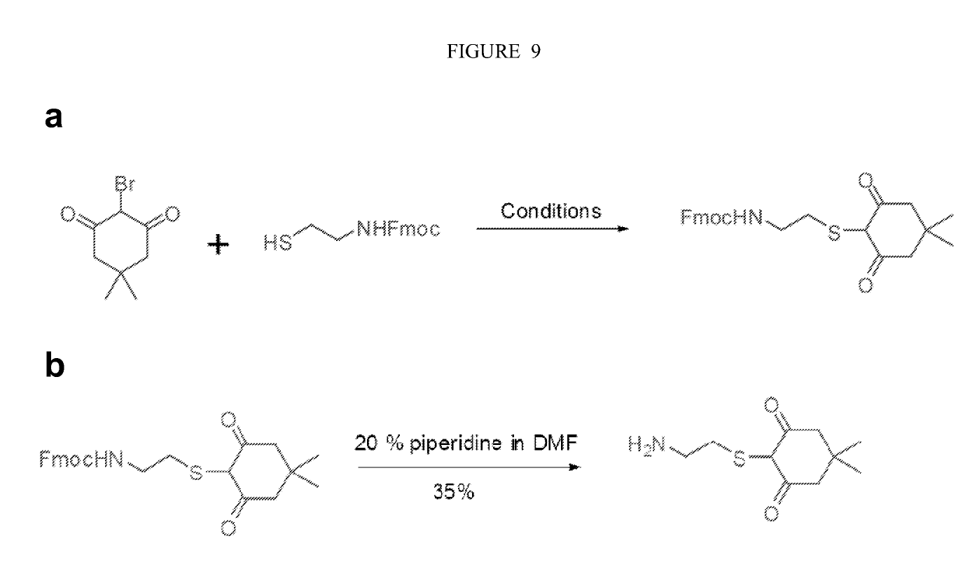

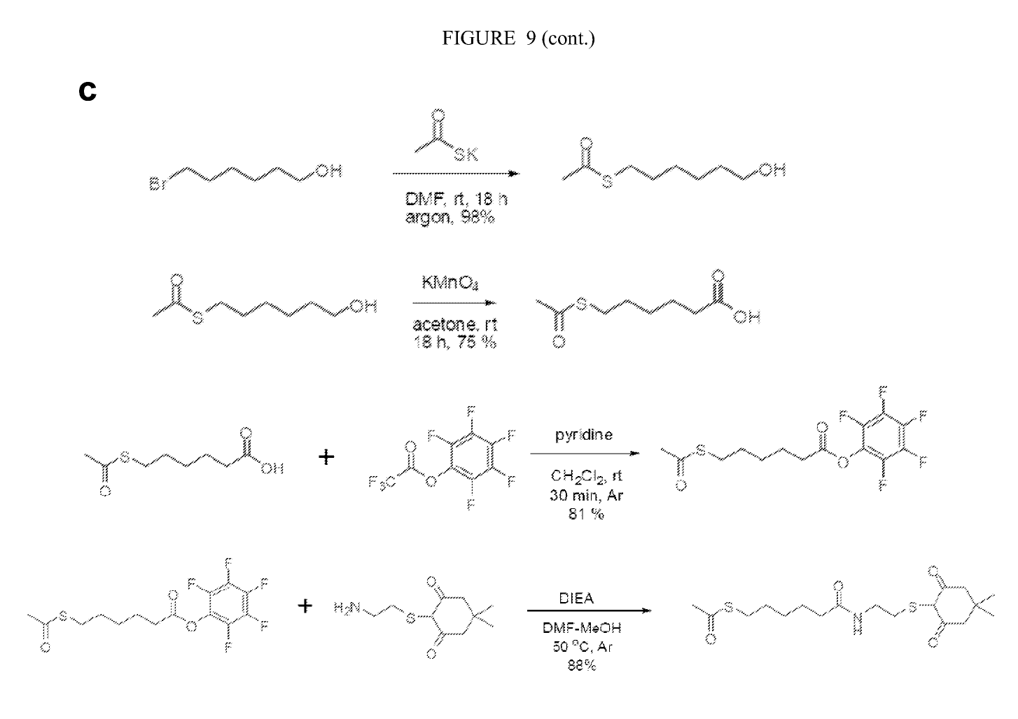

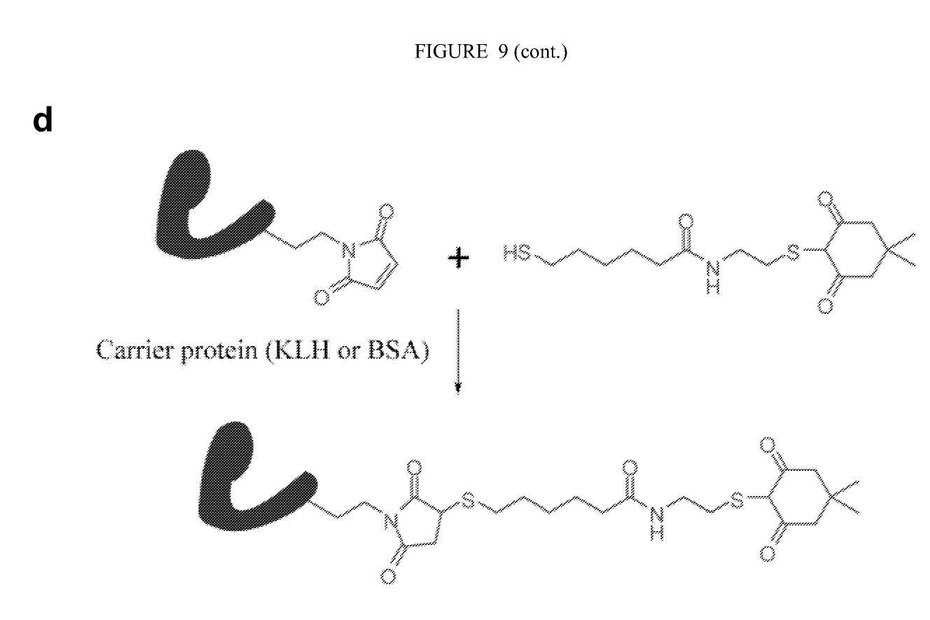

In some embodiments the present invention provides antibodies that recognize oxidative cysteine PTMs independently of protein context. Exemplary antibodies were developed using oxidized cysteine analog haptens conjugated to a carrier protein (KLH or BSA). A Dimedone thioether hapten was synthesized (SEE FIGS. 8A and 8B) and subsequently reacted with a mercaptolinker (SEE FIG. 8C) to yield a hapten capable of conjugation to the carrier protein (SEE FIG. 8D). Animals were subsequently immunized with the hapten-carrier protein conjugate and antibodies were harvested and purified (SEE FIG. 9).

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

### REFERENCES

The following references are herein incorporated by reference in their entireties.
1. Storz, P. (2006) Reactive oxygen species-mediated mitochondria-to-nucleus signaling: a key to aging and radical-caused diseases, Sci STKE 2006, re3.
2. Rhee, S. G. (2006) Cell signaling. H202, a necessary evil for cell signaling, Science 312, 1882-1883.
3. Lambeth, J. D. (2004) NOX enzymes and the biology of reactive oxygen, Nat Rev Immunol 4, 181-189.
4. Stone, J. R., and Yang, S. (2006) Hydrogen peroxide: a signaling messenger, Antioxid Redox Signals, 243-270.
5. Winterbourn, C. C., and Metodiewa, D. (1999) Reactivity of biologically important thiol compounds with superoxide and hydrogen peroxide, Free Radic Biol Med 27, 322-328.
6. Hess, D. T., Matsumoto, A., Kim, S. O., Marshall, H. E., and Stamler, J. S. (2005) Protein S-nitrosylation: purview and parameters, Nat Rev Mol Cell Biol 6, 150-166.
7. Veal, E. A., Day, A. M., and Morgan, B. A. (2007) Hydrogen peroxide sensing and signaling, Mol Cell 26, 1-14.
8. Zheng, M., Aslund, F., and Storz, G. (1998) Activation of the OxyR transcription factor by reversible disulfide bond formation, Science 279, 1718-1721.
9. Delaunay, A., Pflieger, D., Barrault, M. B., Vinh, J., and Toledano, M. B. (2002) A thiol peroxidase is an H202 receptor and redox-transducer in gene activation, Cell 111, 471481.
10. Tonks, N. K. (2005) Redox redux: revisiting PTPs and the control of cell signaling, Cell 121, 667-670.
11. Giannoni, E., Buricchi, F., Raugei, G., Ramponi, G., and Chiarugi, P. (2005) Intracellular reactive oxygen species activate Src tyrosine kinase during cell adhesion and anchorage-dependent cell growth, l'v101 Cell Biol 25, 6391-6403.
12. Cook-Mills, J. M. (2006) Hydrogen peroxide activation of endothelial cell-associated MMPs during VCAM-1 -dependent leukocyte migration, Cell Mol Biol (Noisy-le-grand) 52,8-16.
13. Nobes, C. D., and Hall, A. (1995) Rho, rac, and cdc42 GTPases regulate the assembly of multimolecular focal complexes associated with actin stress fibers, lamellipodia, and filopodia, CeIISl, 53-62.
14. Wu, R. F., Xu, Y. C., Ma, Z., Nwariaku, F. E., Sarosi, G. A., Jr., and Terada, L. S.(2005) Subcellular targeting of oxidants during endothelial cell migration, J Cell Biol 171, 893904.
15. Salmeen, A., Andersen, J. N., Myers, M., Meng, T. C., Hinks, J. A., Tonks, N. K., and Barford, D. (2003) Redox regulation of protein tyrosine phosphatase 1B involves a sulphenyl-amide intermediate, Nature 423, 769-773.
16. Eaton, P. (2006) Protein thiol oxidation in health and disease: techniques for measuring disulfides and related modifications in complex protein mixtures, Free Radic Biol Med 40, 1889-1 899.
17. Leichert, L. I., and Jakob, U. (2004) Protein thiol modifications visualized in vivo, PLoS Biol 2, e333.
18. Saurin, A. T., Neubert, H. Brennan, J. P., and Eaton, P. (2004) Widespread sulfenic acid formation in tissues in response to hydrogen peroxide, Proc Natl Acad Sci USA 101,17982-17987.
19. Poole, L. B., Zeng, B. B., Knaggs, S. A., Yakubu, M., and King, S. B. (2005) Synthesis of chemical probes to map sulfenic acid modifications on proteins, Bioconjug Chem 16, 1624-1628.
20. Agard, N. J., Baskin, J. M., Prescher, J. A., Lo, A., and Bertozzi, C. R. (2006) A comparative study of bioorthogonal reactions with azides, ACS Chem Biol 1, 644-648.
21. Fraenkel-Conrat, H. (1955) The reaction of tobacco mosaic virus with iodine, J Biol Chem 217,373-381.
22. van Montfort, R. L., Congreve, M., Tisi, D., Carr, R., and Jhoti H., (2003) Oxidation state of the active-site cysteine in protein tyrosine phosphatase 1B, Nature 423, 773-777.
23. Benitez, L. V., and Allison, W. S. (1974) The inactivation of the acyl phosphatase activity catalyzed by the sulfenic acid form of glyceraldehyde 3-phosphate dehydrogenase by dimedone and olefins, J Biol Chem 249, 6234-6243.
24. Reynaert, N. L., van der Vliet, A., Guala, A. S., McGovern, T., Hristova, M., Pantano, C., Heintz, N. H., Heim, J., Ho, Y. S., Matthews, D. E., Wouters, E. F., and JanssenHeininger, Y. M. (2006) Dynamic redox control of NF-kappaB through glutaredoxinregulated S-glutathionylation of inhibitory kappaB kinase beta, Proc Natl Acad Sci USA 103, 13086-13091.
25. Claiborne, A., Yeh, J. I., Mallett, C., Luba, J., Crane, E. J., 3rd, Charrier, V., and Parsonage, D. (1999) Protein-sulfenic acids: diverse roles for an unlikely player in enzyme catalysis and redox regulation, Biochemistry 38, 15407-15416.
26. Hamann, M., Zhang, T., Hendrich, S., and Thomas, J. A. (2002) Quantitation of protein sulfinic and sulfonic acid, irreversibly oxidized protein cysteine sites in cellular proteins, Methods Enzymol348, 146-156.
27. Biteau, B., Labarre, J., and Toledano, M. B. (2003) ATP-dependent reduction of cysteine-sulphinic acid by S. cerevisiae sulphiredoxin, Nature 425, 980-984.
28. Fu, X., Kassim, S. Y., Parks, VV. C., and Heinecke, J. VV. (2001) Hypochlorous acid oxygenates the cysteine switch domain of pro-matrilysin (MMP-7). A mechanism for matrix metalloproteinase activation and atherosclerotic plaque rupture by myeloperoxidase, J Biol Chem 276, 41279-41287.
29. Murakami, T., Nojiri, M., Nakayama, H., Odaka, M., Yohda, M., Dohmae, N., Takio, K., Nagamune, T., and Endo, I. (2000) Post-translational modification is essential for catalytic activity of nitrile hydratase, Protein Sci 9, 1024-1030.
30. Ritchie, C. D., Saltiel, J. D., and Lewis, E. S. (1961) The reaction of diazonium salts with nucleophiles, Journal of the American Chemical Society 83,4601-4605.
31. Schlick, T. L., Ding, Z. ,Kovacs, E. W. and Francis, M. B. (2005) Dual-surface modification of the tobacco mosaic virus, J Am Chem Soc 127, 3718-3723.
32. Lin, W. S., Armstrong, D. A. and Gaucher, G. M. (1975) Formation and repair of papain sulfenic acid, Can J Biochem 53,298-307.
33. Jacob, C., Giles, G. I., Giles, N. M., and Sies, H. (2003) Sulfur and selenium: the role of oxidation state in protein structure and function, Angew Chem Int Ed Engl 42, 47424758.
34. Barford, D., Keller, J. C., Flint, A. J., and Tonks, N. K. (1994) Purification and crystallization of the catalytic domain of human protein tyrosine phosphatase 1 B expressed in Escherichia coli, J Mol Biol 239, 726-730.
35. Gu, Z., Kaul, M., Van, B., Kridel, S. J., Cui, J., Strongin, A., Smith, J. W., Liddington, R. C., and Lipton, S. A. (2002) S-nitrosylation of matrix metalloproteinases: signaling pathway to neuronal cell death, Science 297, 1186-1190.
36. Ellis, H. R., and Poole, L. B. (1997) Novel application of 7-chloro-4-nitrobenzo-2-oxal,3- diazole to identify cysteine sulfenic acid in the AhpC component of alkyl hydroperoxide reductase, Biochemistry 36, 15013-15018.
37. Woo, H. A., Kang, S. W., Kim, H. K., Yang, K. S., Chae, H. Z, and Rhee, S. G. (2003) Reversible oxidation of the active site cysteine of peroxiredoxins to cysteine sulfinic acid. Immunoblot detection with antibodies specific for the hyperoxidized cysteinecontaining sequence, J Biol Chem 278,47361-47364.
38. Hoffhines, A. J., Damoc, E., Bridges, K. G., Leary, J. A., and Moore, K. L. (2006) Detection and purification of tyrosine-sulfated proteins using a novel anti-sulfotyrosine monoclonal antibody, J Biol Chem 281,37877-37887.
39. Ross, A. H., Baltimore, D., and Eisen, H. N. (1981) Phosphotyrosine-containing proteins isolated by affinity chromatography with antibodies to a synthetic hapten, Nature 294, 654-656.
40. Alexander, J., del Guercio, M. F., Maewal, A., Qiao, L., Fikes, J., Chesnut, R. W., Paulson, J., Bundle, D. R., DeFrees, S., and Sette, A. (2000) Linear PADRE T helper epitope and carbohydrate B cell epitope conjugates induce specific high titer IgG antibody responses, J. Immunol. 164, 1625-1633.
41. Rush, J., Moritz, A., Lee, K. A., Guo, A., Goss, V. L., Spek, E. J., Zhang, H., Zha, X. M., Polakiewicz, R. D., and Comb, M. J. (2005) Immunoaffinity profiling of tyrosine phosphorylation in cancer cells, Nat Biotechnol 23, 94-101.
42. Saxon, E., and Bertozzi, C. R. (2000) Cell surface engineering by a modified Staudinger reaction, Science 287,2007-2010.
43. Hang, H. C., Geutjes, E. J., Grotenbreg, G., Poiiington, A. M., Bijimakers, M. J., and Ploegh, H. L. (2007) Chemical probes for the rapid detection of Fatty-acylated proteins in Mammalian cells, J Am Chem Soc 129,2744-2745.
44. Hang, H. C., Loureiro, J., Spooner, E., van der Velden, A. 'lV., Kim, Y. ˜v1., Pollington, A. M., Maehr, R., Stambach, M. N., and Ploegh, H. L. (2006) Mechanism-based probe for the analysis of cathepsin cysteine proteases in living cells, ACS Chem Biol 1, 713-723.
45. Coon, J. J., Syka, J. E., Shabanowitz, J., and Hunt, D. F. (2005) Tandem mass spectrometry for peptide and protein sequence analysis, Biotechniques 38, 519, 521
46. Ishihama, Y. (2005) Proteomic LC-MS systems using nanoscale liquid chromatography with tandem mass spectrometry, J Chromatogr A 1067, 73-83.
47. Kamata, H., Honda, S., Maeda, S., Chang, L., Hirata, H., and Karin, M. (2005) Reactive oxygen species promote TNFalpha-induced death and sustained JNK activation by inhibiting MAP kinase phosphatases, Cell 120, 649-661.
48. Oda, Y., Nagasu, T., and Chait, B. T. (2001) Enrichment analysis of phosphorylated proteins as a tool for probing the phosphoproteome, Nat Biotechnol 19, 379-382.
49. Zhou, H., Watts, J. D., and Aebersold, R. (2001) A systematic approach to the analysis of protein phosphorylation, Nat Biotechnol 19, 3 75-3 78.
50. Mann, M. (2006) Functional and quantitative proteomics using SILAC, Nat Rev Mol Cell Biol 7, 952-958.
51. Griendling, K., and FitzGerald, G. A. (2003) Oxidative stress and cardiovascular injury: Part I. basic mechanisms and in vivo monitoring of ROS, Circulation 108, 191 1916.
52. Griendling, K. K., and FitzGerald, G. A. (2003) Oxidative stress and cardiovascular injury: Part 1/: animal and human studies, Circulation 108, 2034-2040.

We claim:

1. A composition comprising a compound of Formula I:
C L-H
wherein C is a chemoselective moiety configured to specifically bind a single post-translationally-modified cysteine oxidative state, wherein said single post-translationally-modified cysteine oxidative state is selected from thiol, cysteine-sulfenic acid, cysteine-sulfinic acid, and cysteine-sulfonic acid, wherein said binding is capable of occurring in vivo and in vitro, and wherein said binding occurs in a context independent manner; wherein L is a linker moiety; and wherein H is a handle moiety.

2. The composition of claim 1, wherein said handle moiety comprises an azide group.

3. The composition of claim 1, wherein said compound is cell permeable.

4. The composition of claim 1, wherein said chemoselective moiety covalently and specifically binds cysteine-sulfenic acid.

5. The composition of claim 1, wherein said chemoselective moiety covalently and specifically binds cysteine-sulfinic acid.

6. The composition of claim 1, wherein said chemoselective moiety covalently and specifically binds cysteine-thiol.

7. The composition of claim 1, wherein said chemoselective moiety comprises 3,5-dihydroxybenzaldehyde.

8. The composition of claim 1, wherein said chemoselective moiety comprises para-diazonium benzaldehyde.

9. The composition of claim 1, wherein said chemoselective moiety comprises a diazonium-containing compound

10. The composition of claim 1, wherein said handle comprises a ligand, antigen, or reactive group for a secondary binding agent.

11. A method comprising:
a) providing:
i) a chemoselective agent capable of specifically binding a single post-translationally-modified cysteine oxidative state in a context independent manner, and
ii) a sample containing the amino acid cysteine in one or more oxidative states, wherein said sample may be in vivo or in vitro, wherein said cysteines may be present in the sample as single amino acids, part of a peptide, part of a polypeptide, or part of a protein;
b) administering said chemoselective agent to said sample; and
c) reacting of said single post-translationally-modified cysteine-oxidative state with said chemoselective agent to form a chemical bond, wherein said reaction is non-specific with regards to the peptides, polypeptides, or proteins.

12. The method of claim 11, further comprising:
d) providing a secondary agent, wherein said secondary agent;
e) administering said secondary agent to said sample; and
f) interacting said secondary agent with said chemoselective agent is configured to bind to said chemoselective agent, wherein said interaction comprises covalent bonding or specific molecular recognition, wherein said interaction occurs specifically between said secondary agent and said chemoselective agent, wherein said interaction occurs non-specifically with regards to the peptides, polypeptides, or proteins.

13. The method of claim 10, further comprising:
g) providing a tertiary agent;
h) administering said tertiary agent to said sample; and
i) forming a stable interaction between said secondary agent and said tertiary agent.

14. The method of claim 13, wherein said chemoselective agent comprises an azido handle.

15. The method of claim 14, wherein said secondary agent comprises phosphene-biotin (P-Biotin).

16. The method of claim 15, wherein said tertiary agent comprises streptavidin.

17. The method of claim 16, wherein said streptavidin is conjugated to horseradish peroxidase.

18. The method of claim 12, wherein said chemoselective agent comprises dimedone.

19. The method of claim 18, wherein the secondary agent is an antibody, wherein said antibody is capable of specific molecular recognition of dimedone, wherein a stable interaction is formed between said antibody and said dimedone upon said specific molecular recognition.

20. The method of claim 11, wherein said single post-translationally-modified cysteine oxidative state comprises a thiol, sulfenic acid, sulfinic acid, or sulfonic acid.
